(19) United States
(12) Patent Application Publication
(10) Pub. No.: US 2026/0014014 A1
(43) Pub. Date: Jan. 15, 2026

(54) Nasal Device For Improving Nasal Breathing

(71) Applicant: R. Joseph Magness, Orem, UT (US)

(72) Inventor: R. Joseph Magness, Orem, UT (US)

(21) Appl. No.: 19/336,497

(22) Filed: Sep. 23, 2025

Related U.S. Application Data
(63) Continuation of application No. 17/027,668, filed on Sep. 21, 2020, now Pat. No. 12,419,775.
(60) Provisional application No. 62/903,170, filed on Sep. 20, 2019.
(60) Provisional application No. 62/959,720, filed on Jan. 10, 2020.

(51) Int. Cl. A61F 5/08; B29C 64/393; B29L 31/00; B33Y 50/02; B33Y 80/00

(52) U.S. Cl. CPC A61F 5/08; B29C 64/393; B33Y 50/02; B33Y 80/00; B29L 2031/753

(57) ABSTRACT

A nasal device may include a standard shaped to extend over an exterior portion of a user's nose from a first nasal opening, across flexible nasal tissues, to a second nasal opening. The standard may have a first end and a second end. A first regulator may be connected to the first end and extend therefrom into the first nasal opening. The first regulator may be shaped to engage the flexible tissues and regulate their position by pushing them toward an underside of the standard. A second regulator may be connected to the second end and extend therefrom into the second nasal opening. The second regulator may also be shaped to engage the flexible tissues and regulate their position by pushing them toward the underside of the standard.

### 1. RELATED APPLICATIONS

This application is a continuation of U.S. patent application Ser. No. 17,027,668 filed Sep. 21, 2020 (now U.S. Pat. No. 12,419,775), which claims the benefit of U.S. Provisional Patent Application Ser. No. 62,903,170 filed Sep. 20, 2019 and U.S. Provisional Patent Application Ser. No. 62,959,720 filed Jan. 10, 2020. Each of the foregoing patent applications is hereby incorporated by reference.

### BACKGROUND

### 2. Field of the Invention

This invention relates to nasal devices and, more particularly, to novel systems and methods for improving nasal breathing of a human user.

### 3. Background Art

An external nasal dilator is typically secured to the skin of the nose of a human user via an adhesive. Once applied to the nose, an external nasal dilator may lift the outer wall tissues of the nostrils. This may dilate the nasal passage and reduce resistance to airflow during nasal breathing. One disadvantage of such an external nasal dilator is that the skin of the user must be substantially oil free for sufficient adhesion to occur. Accordingly, what is needed is a device that improves nasal breathing without requiring adhesive.

### BRIEF DESCRIPTION OF THE DRAWINGS

The features of the present invention will become apparent from the following description and appended claims, taken in conjunction with the accompanying drawings. Understanding that these drawings depict only typical embodiments of the invention and are, therefore, not to be considered limiting of its scope, the invention will be described with additional specificity and detail through use of the accompanying drawings in which:

FIG. 1 is a front view of one embodiment of a nasal device in accordance with the present invention;

FIG. 2 is a front view of the nasal device of FIG. 1 applied to a human user;

FIG. 3 is a side view of the nasal device of FIG. 1 applied to a human user;

FIG. 4 is a cross-sectional view of a portion of the nasal device of FIG. 1 applied to a human user;

FIG. 5 is a block diagram of one embodiment of a method in accordance with the present invention;

FIG. 6 is a front view of an alternative embodiment of a nasal device in accordance with the present invention;

FIG. 7 is a front view of another alternative embodiment of a nasal device in accordance with the present invention;

FIG. 8 is a front view of another alternative embodiment of a nasal device in accordance with the present invention;

FIG. 9 is a block diagram of an alternative embodiment of a method in accordance with the present invention;

FIG. 10 is a block diagram of another alternative embodiment of a method in accordance with the present invention;

FIG. 11 is a perspective view of one embodiment of a tool in accordance with the present invention with one end of the tool forming an internal nasal prop and the opposite end of the tool forming a measurement device (e.g., a thickness gauge);

FIG. 12 is another perspective view of the tool of FIG. 11;

FIG. 13 is a first side view of the tool of FIG. 11;

FIG. 14 is a second, opposite side view of the tool of FIG. 11;

FIG. 15 is a bottom view of the tool of FIG. 11;

FIG. 16 is a top view of the tool of FIG. 11;

FIG. 17 is a first end view of the tool of FIG. 11;

FIG. 18 is a second, opposite end view of the tool of FIG. 11;

FIG. 19 is a perspective view of an alternative embodiment of a nasal device in accordance with the present invention;

FIG. 20 is another perspective view of the nasal device of FIG. 19;

FIG. 21 is a front view of the nasal device of FIG. 19;

FIG. 22 is a back view of the nasal device of FIG. 19;

FIG. 23 is a right side view of the nasal device of FIG. 19;

FIG. 24 is a left side view of the nasal device of FIG. 19;

FIG. 25 is a top view of the nasal device of FIG. 19;

FIG. 26 is a bottom view of the nasal device of FIG. 19;

FIG. 27 is a perspective view of a right stock portion of the nasal device of FIG. 19;

FIG. 28 is a perspective view of a left stock portion of the nasal device of FIG. 19;

FIG. 29 is a right stock portion in large, medium, and small sizes in accordance with the present invention;

FIG. 30 is a block diagram of another alternative embodiment of a method in accordance with the present invention;

FIG. 31 is a cross-sectional view of a portion of another alternative embodiment of a nasal device in accordance with the present invention;

FIG. 32 is a front view of another alternative embodiment of a nasal device in accordance with the present invention; and

FIG. 33 is a front view of another alternative embodiment of a nasal device in accordance with the present invention.

### DETAILED DESCRIPTION

It will be readily understood that the components of the present invention, as generally described and illustrated in the drawings herein, could be arranged and designed in a wide variety of different configurations. Thus, the following more detailed description of the embodiments of the system and method of the present invention, as represented in the drawings, is not intended to limit the scope of the invention, as claimed, but is merely representative of various embodiments of the invention. The illustrated embodiments of the invention will be best understood by reference to the drawings, wherein like parts are designated by like numerals throughout.

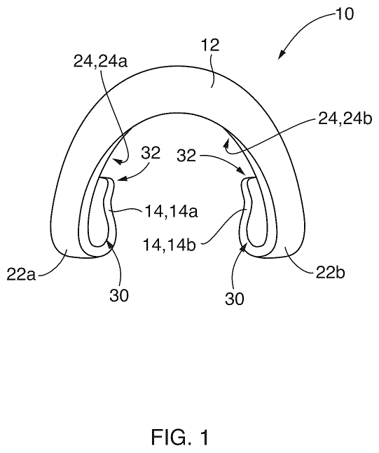

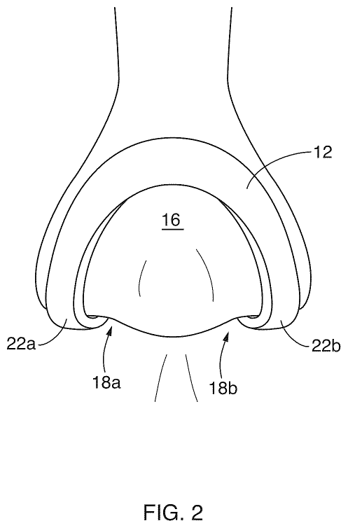

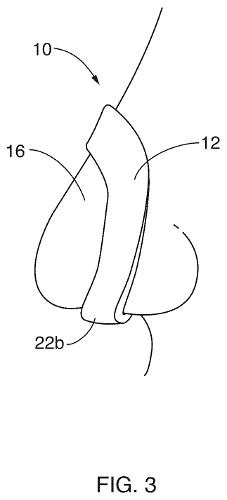

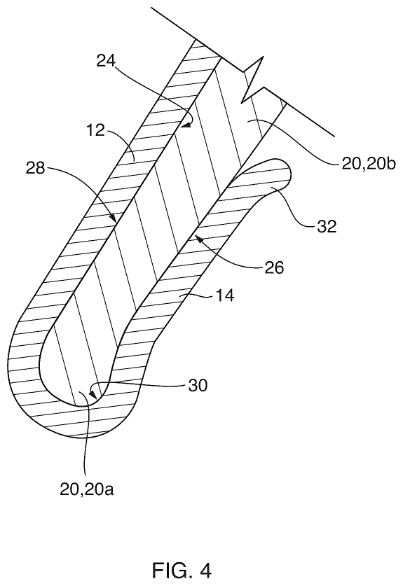

Referring to FIGS. 1-4, a nasal device 10 in accordance with the present invention may include a standard 12 and one or more regulators 14 (e.g., two regulators 14). A standard 12 may be sized and shaped to extend over a portion of an exterior of a nose 16 of a human user. In certain embodiments, a standard 12 may be sized and shaped to extend from one nasal opening 18a upward over the flexible tissues 20 (e.g., the greater alar cartilage, lateral cartilage, surrounding skin, etc.) of one side of the nose, over the cartilage of septum, downward over the flexible tissues 20 of the other side of the nose, and to the other nasal opening 18b.

A standard 12 may have two ends (e.g., a first end 22a and a second end 22b). When applied to the nose 16 of a user, a first end 22a of a standard 12 may be positioned proximate a first nasal opening 18a and a second end 22b of the standard 12 may be positioned proximate a second nasal opening 18b.

In selected embodiments, a first regulator 14a may connect to and extend from a first end 22a of a standard 12. A second regulator 14b may connect to and extend from a second end 22b of a standard 12. When applied to the nose 16 of a user, a first regulator 14a may extend into a first nasal opening 18a and a second regulator 14b may extend into a second nasal opening 18b.

An underside 24 of a standard 12 may define an improved or optimal location of the flexible tissues 20 that form the outer wall of a corresponding nostril. For example, an underside 24a corresponding to one half of a standard 12 may define an improved or optimal location of the flexible tissues 20 that form the outer wall of a first nostril. Accordingly, when the flexible tissues 20 that form the outer wall of the first nostril abut that underside 24a, the first nostril may be positioned for improved or optimized nasal breathing. Similarly, an underside 24b corresponding to the other half of the standard 12 may define an improved or optimal location of the flexible tissues 20 that form the outer wall of a second nostril. Accordingly, when the flexible tissues 20 that form the outer wall of the second nostril abut that underside 24b, the second nostril may be positioned for improved or optimized nasal breathing.

Regulators 14 may be sized, shaped, and positioned to hold certain flexible tissues 20 of a nose 16 to a corresponding standard 12. That is, a regulator 14 may push against an inner surface 26 of the flexible tissues 20 until an outer surface 28 of the flexible tissues 20 abuts an underside 24 of a standard 12. Accordingly, when the flexible tissues 20 that form the outer wall of a first nostril are pushed by a first regulator 14a against an underside 24a corresponding to one half of a standard 12, then the first nostril may be positioned for improved or optimized nasal breathing. Similarly, when the flexible tissues 20 that form the outer wall of a second nostril are pushed by a second regulator 14b against an underside 24b corresponding to the other half of a standard 12, then the second nostril may be positioned for improved or optimized nasal breathing.

In selected embodiments, the flexible tissue 20a forming or defining a nasal opening 18 may be somewhat bulbous or thicker than the flexible tissue 20b further up the nose 16. In such embodiments, one or more corresponding regulators 14 may define a slightly larger cavity 30 to accommodate this bulbous or thicker flexible tissue 20b. In certain embodiments, the interaction between the bulbous or thicker flexible tissue 20b and a corresponding larger cavity 30 may create a detent of sorts that may tend to hold a nasal device 10 in a fully installed or seated configuration. In certain embodiments, the tip 32 of a regulator 14 may be curved, smoothed, ramped, or a combination or sub-combination thereof to make application of the nasal device 10 to a user a painless and easy process.

A nasal device 10 in accordance with the present invention may be formed of a material having sufficient thickness and rigidity to flex the flexible tissues 20 rather than be flexed by those tissues 20. Accordingly, in use, the flexible tissues 20 may conform to the standard 12 while the standard 12 remains substantially unchanged. In selected embodiments, a nasal device 10 may comprise a standard 12 and two regulators 14a, 14b monolithically formed as a single piece of polymeric material.

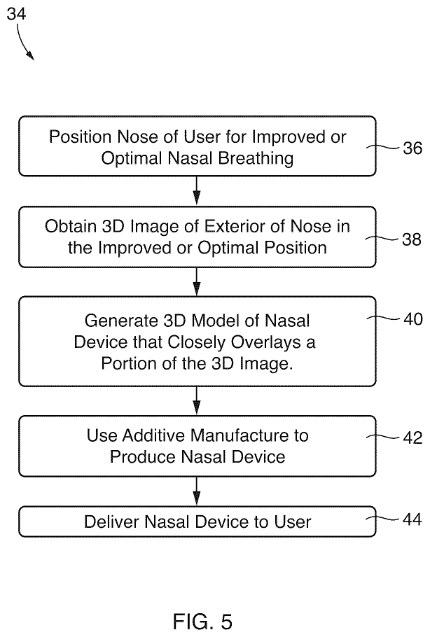

Referring to FIG. 5, a nasal device 10 in accordance with the present invention may be manufactured in any suitable manner. In selected embodiments, a standard 12 and two regulators 14a, 14b may be made as a monolithic unit using three-dimensional printing. This may enable a nasal device 10 to be custom made for each user. Custom manufacture of a nasal device 10 in accordance with the present invention may ensure that the nasal device 10 is both comfortable to wear and effective (e.g., maximally effective) for each user.

For example, in one method 34, a nose 16 of a user may be positioned 36 for improved or optimal breathing. This may involve examining certain internal features of the nostrils of the user and deflecting certain flexible tissues 20 of nose 16 of the user so as to improve or optimize the flow of air through one or both nostrils. In selected embodiments, once the improved or optimized position is identified, steps may be taken to hold the flexible tissues 20 of the nose 16 in the improved or optimized position(s). This may be accomplished by inserting filler or packing material into one or both nostrils.

Once the nose 16 of the user is properly positioned, a three-dimensional model of the exterior of a user's nose may be obtained 38 or generated via a digital scan. The three-dimensional model of the nose may be used to create 40 a three-dimensional model of a nasal device 10. Specifically, a standard 12 may be shaped to match the improved or optimized position of the nose 16 of a user. Accordingly, a standard 12 may track (e.g., closely follows selected contours of) the nose 16 so positioned. Moreover, one or more regulators 14 may be digitally defined so as to appropriately fit and extend into corresponding nasal openings 18.

The gap between a standard 12 and a regulator 14, the size and shape of a cavity 30 defined by a regulator 14, the length of a regulator 14, and the like may be customized. That is, they may be based on measurements collected from the corresponding user. Alternatively, one or more such features may be determined based on certain average or expected measurements. Accordingly, in certain embodiments, a computer-aided drafting (CAD) application or the like may be used to import one or more regulators 14 into the three dimensional model. The regulators 14 may be a predefined, standard part. Accordingly, they may be imported and located as desired.

Once a standard 12 and one or more regulators 14 are properly positioned within a digital three dimensional model, the interfaces between the various components may be filled and/or smoothed so as to provide the desired comfort and functionality. The completed digital three dimensional model may then be printed 42 in a polymeric material using a three-dimensional printing process. The resulting nasal device 10 may then be delivered 44 to the corresponding user.

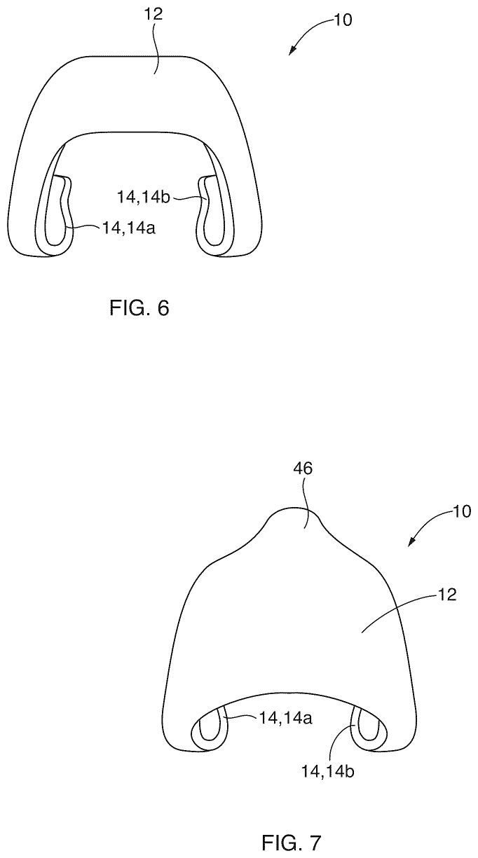

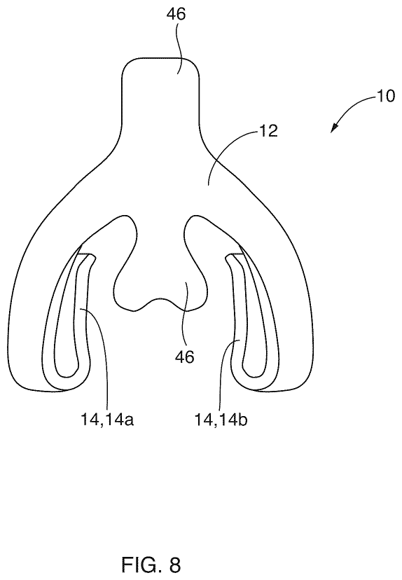

Referring to FIGS. 6-8, a standard 12 in accordance with the present invention may have any suitable shape. Accordingly, a standard 12 may cover or closely track more or less of the exterior of a nose 16 in an improved or optimized position. Greater coverage of the nose 16 may enable or support more shaping of or control over the flexible tissues 20. Lesser coverage of the nose 16 may make the device 10 lighter and enable the skin to interact more normally with the surrounding air (e.g., “breathe” better).

In selected embodiments, a standard 12 may include one or more extensions 46. The one or more extensions 46 may be sized and positioned so as to align with and extend along a ridge of a nose 16. Accordingly, one or more extensions 46 may tend to stabilize a standard 12 with respect to a nose 16.

Regulators 14 in accordance with the present invention may have any suitable length. Accordingly, regulators 14 may extend any suitable distance into the nostrils of a user. In general, a regulator 14 may extend sufficiently to hold corresponding flexible tissue 20 to a standard 12 and insufficiently to become a nuisance to or uncomfortable for the user.

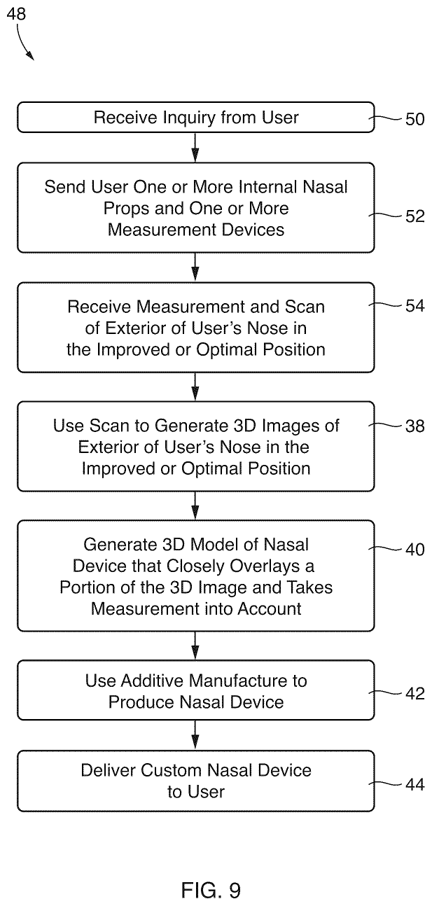

Referring to FIG. 9, in selected embodiments, a method 48 may enable a manufacturer of nasal devices 10 in accordance with the present invention to serve remote users (e.g., users that cannot easily travel to the manufacturer for measurement). Such a method 48 may include receiving 50 an inquiry from a user (e.g., from the user directly or from a healthcare professional associated with the user). In response to the inquiry, a manufacturer may send 52 the user (or a healthcare professional associated with the user) one or more internal nasal props and/or one or more measurement devices. Thereafter, the manufacturer may receive 54 from the user (or a healthcare professional associated with the user) one or more measurements of the thickness of the flexible tissues 20 of the user's nose 16 and/or a digital scan of the exterior of the user's nose 16 in an improved or optimal position for breathing. Accordingly, using the measurement and/or scan, a manufacturer may take steps 38, 40, 42 to deliver 44 (e.g., ship) a custom nasal device 10 to the user.

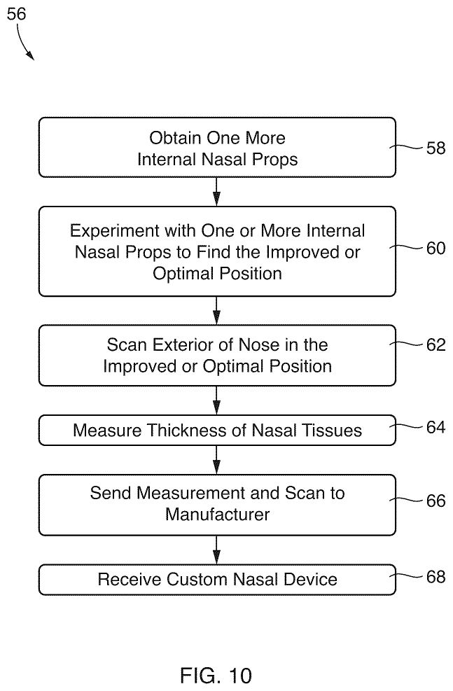

Referring to FIG. 10, in certain embodiments, a method 56 may enable a user to receive a nasal device 10 in accordance with the present invention from a remote manufacturer (e.g., a manufacturer to which the user cannot easily travel for measurement). Such a method 56 may include obtaining 58 one or more internal nasal props. Accordingly, a user may experiment 60 with one or more internal nasal props to find an improved or optimal position for breathing.

For example, in selected embodiments, a user may insert a first internal nasal prop into a first nostril opening and position it therewithin to push and hold (e.g., prop) the flexible tissues 20 outward. A similar process may be followed for a second nostril. The one or more internal nasal props may have various sizes and/or shapes (e.g., pairs of different sizes and/or shapes). Accordingly, a user may experiment 60 with different internal nasal props and/or positioning of those props until the user has identified an improved or optimal position of the flexible tissues 20 corresponding to both nostrils. The improved or optimal position may be a comfortable position wherein the flexible tissues 20 are deflected outward sufficiently to provide an improved or optimal nasal breathing experience for the corresponding user.

Once a user has identified an improved or optimal position of the flexible tissues 20 corresponding to both nostrils, the user may leave the props producing that position in place and then scan 62 the exterior of his or her nose 16. In selected embodiments, a user may use a computerized mobile device (e.g., a smart phone, tablet, or the like) having a camera and running corresponding software to effect the scan. In certain embodiments, such a scan may include the user moving a mobile device during the scan. For example, it may include sweeping a mobile device through a horizontal arc of about 180 degrees from one side of his or her face to the other.

In selected embodiments, a user may measure 64 a thickness (e.g., a distance between an inner surface 26 and an outer surface 28) of the flexible tissues 20 of his or her nose 16. In certain embodiments, a user may use a measurement device to assist in this measuring 64. For example, on or in connection with one or more internal nasal props, a user may obtain one or more gauges of different sizes. By applying different gauges to the flexible tissues 20 of his or her nose 16, a user may determine which gauge fits best. The size indicated on the gauge that fits best may be the thickness of the flexible tissues 20.

Once the scan and the measurement are complete, a user may send 66 the measurement and the scan to a manufacturer. Thereafter, a user may receive 68 a custom nasal device 10 from the manufacturer.

In selected embodiments, an app (e.g., a software application running on a mobile device of a user) may facilitate a method 56 in accordance with the present invention. For example, a user may download an app provided by a manufacturer. Via the app, the user may establish an account and request one or more internal nasal props and/or one or more measurement devices. The app may provide information (e.g., text, videos, etc.) that show a user how to use one or more internal nasal props and/or one or more measurement devices. The app may enable the corresponding mobile device to perform 62 a scan of the exterior of the user's nose 16 once it is in an improved or optimal position. The app may also provide a communication link for sending 66 the scan (e.g., one or more computer files or images), a measurement of the thickness of the flexible tissues 20, payment information, or the like or a combination or sub-combination thereof to the manufacturer.

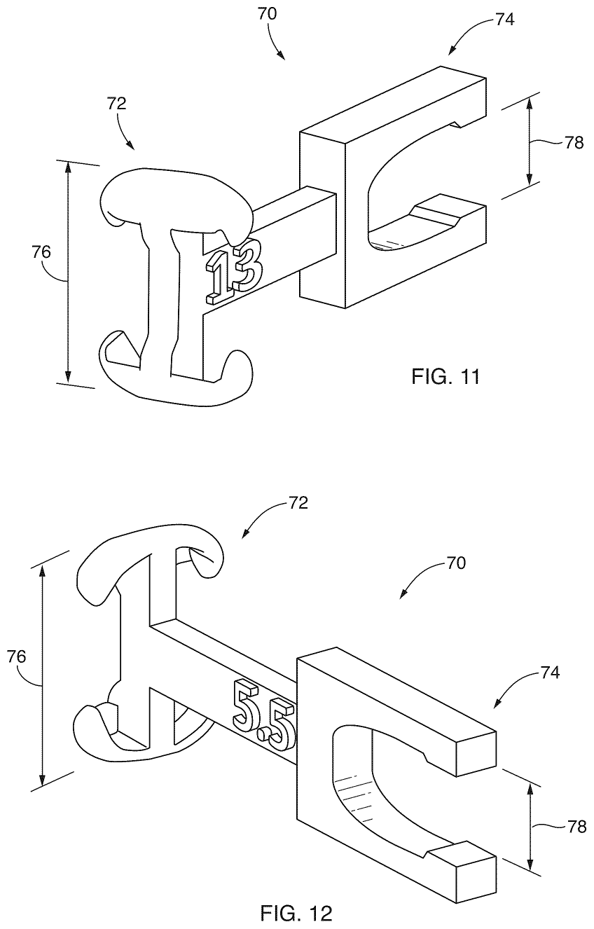

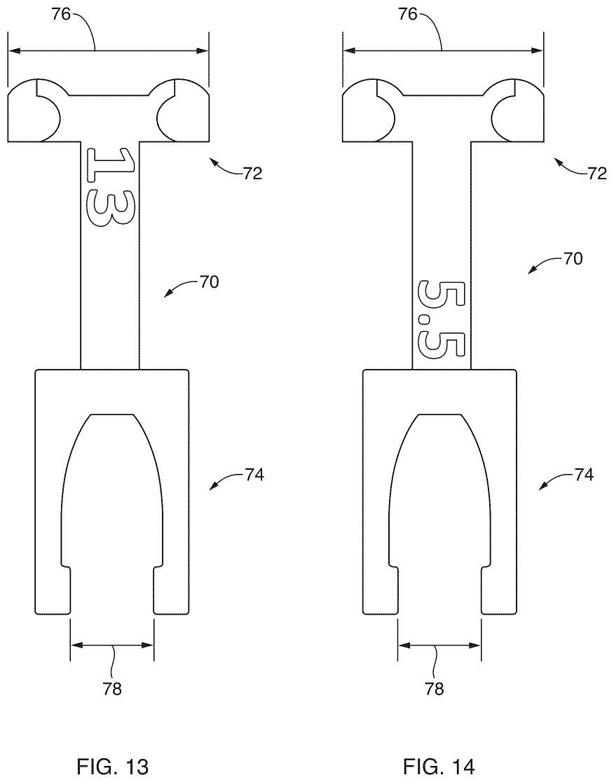

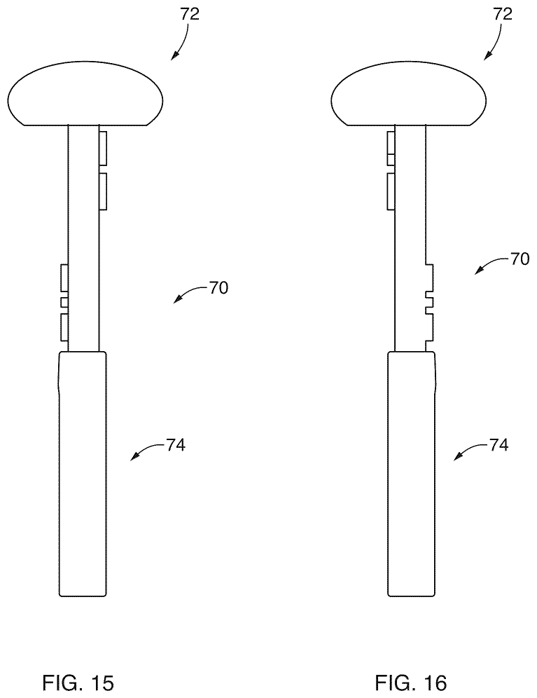

Referring to FIGS. 11-18, a user may receive one or more internal nasal props and/or one or more measurement devices in the form of one or more tools 70 in accordance with the present invention. In selected embodiments, a tool 70 may have an end that forms of is an internal nasal prop 72. Alternatively, or in addition thereto, a tool 70 may have an end that forms or is a measurement device 74 (e.g., a gauge for determining thickness).

In certain embodiments, multiple tools 70 having internal nasal props 72 and/or measurement devices 74 of different sizes may be provided to a user. For example, in the illustrated embodiment, the internal nasal prop 72 has a height 76 of about 13 mm. The height 76 may be indicated on the tool 70 proximate the internal nasal prop 72. Other internal nasal props 72 may have a height 76 that is larger or smaller than 13 mm. The respective heights of those other internal nasal props 72 may be similarly indicated so that a user can distinguish them from each other. Accordingly, a user may experiment 60 to find an improved or optimal position.

In selected embodiments, an internal nasal prop 72 may have rounded exterior surfaces so that it may be comfortably inserted within a nostril of a user. Additionally, an internal nasal prop 72 may have a relatively slim profile so as to not overly restrict airflow through a nostril when inserted therewithin. Accordingly, a user may breathe through his or her nose 16 with an internal nasal prop 72 inserted within each nostril and, thereby, personally experience how the spacing enforced by the internal nasal props affects (e.g., improves) his or her nasal breathing.

In the illustrated embodiment, the measurement device 74 is a thickness gauge having a gap 78 of about 5.5 mm. The specific size of a gap 78 may be indicated on the tool 70 proximate the measurement device 74. Other measurement devices 74 may have a gap 78 that is larger or smaller than 5.5 mm. The respective gaps 78 of those other measurement devices 74 may be similarly indicated so that a user can distinguish them from each other. Accordingly, a user may slide different measurement devices 74 onto the flexible tissues 20 of his or her nose 16 to find one that fits snugly and/or comfortably. When the best fitting measurement device 74 is identified, the user may identify the thickness measurement by reading it on the corresponding tool 70.

In selected embodiments, an array of tools 70 having internal nasal props 72 and/or measurement devices 74 of various sizes (e.g., various heights 76 and/or gaps 78) in accordance with the present invention may be provided 52 to a user or obtained 58 by a user. Accordingly, a user may have what is needed to find an improved or optimal position and/or measure a thickness of the flexible tissues 20 of his or her nose 16.

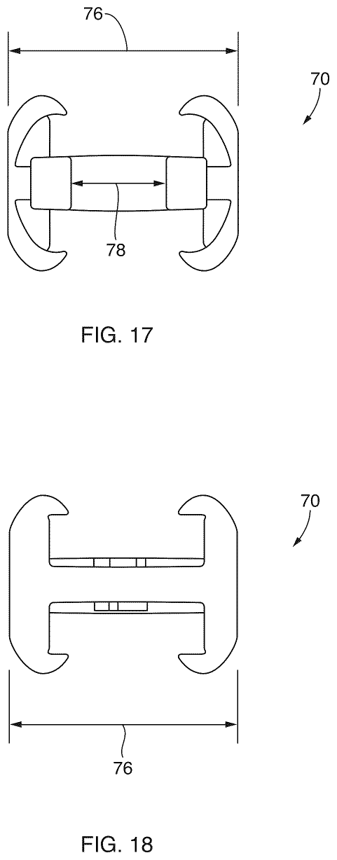

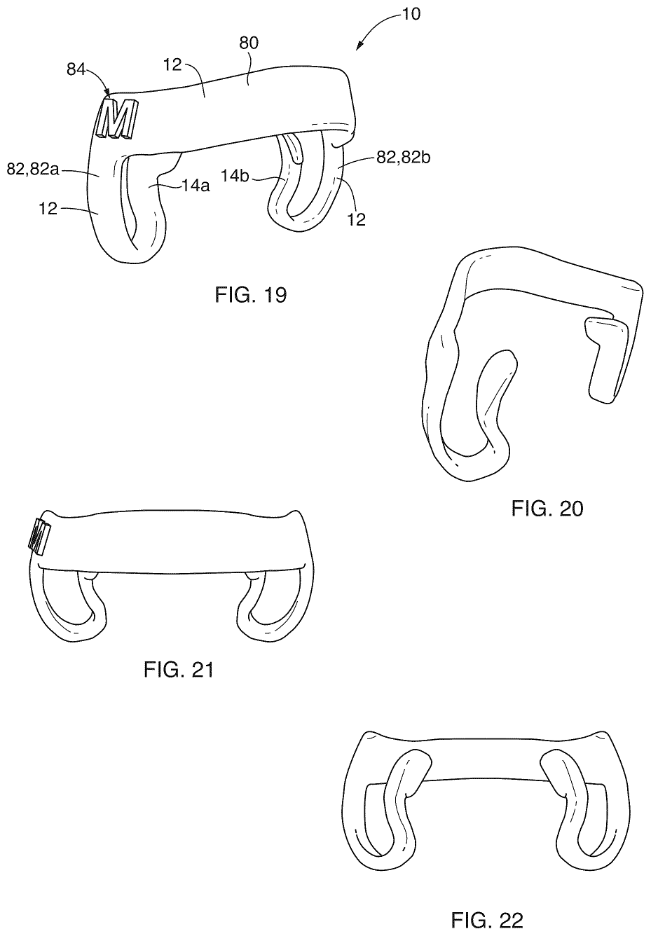

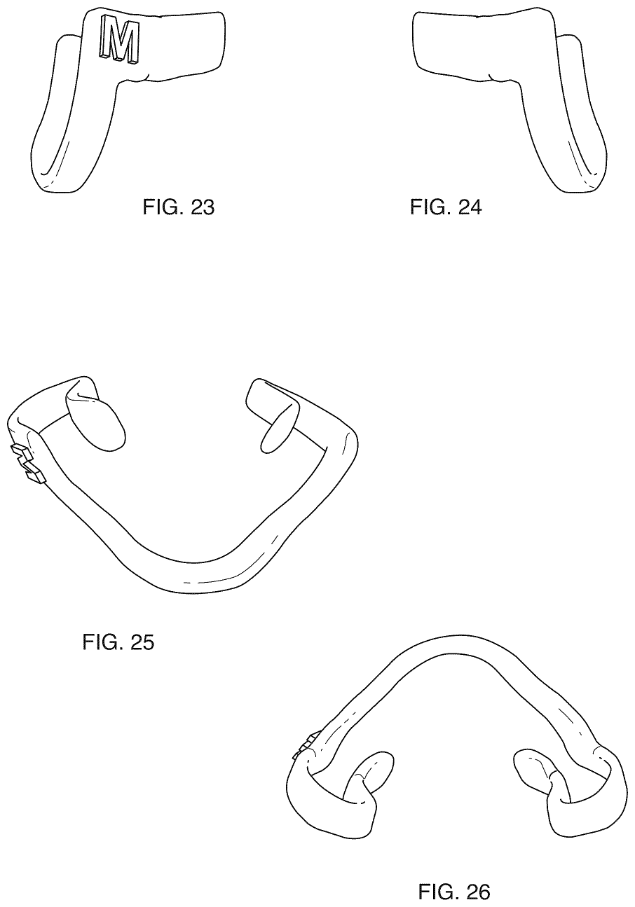

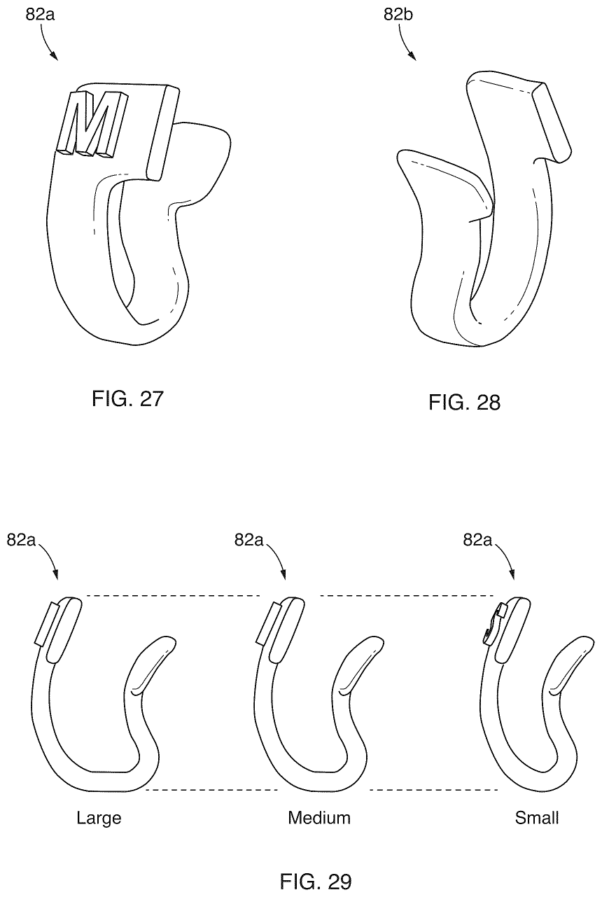

Referring to FIGS. 19-29, in selected embodiments, a nasal device 10 in accordance with the present invention may have a customized portion 80 and one or more stock portions 82. In selected embodiments, a stock portion 82 may comprise part of a standard 12 and part of a regulator 14. A customized portion, on the other hand, may only be part of a standard 12. A customized portion 80 may be customized for a particular user. A stock portion 82 may be standardized and used for various users. In certain embodiments, one stock portion 82 may be connected to each end of a customized portion 80. Accordingly, a customized portion 80 may comprise a bridge extending to connect one stock portion 82 (e.g., a right handed version 82a) a to another stock portion 82 (e.g., a left handed version 82b).

Stock portions 82 may also be available in right handed versions 82a and left handed versions 82b. Accordingly, when a virtual model of a nasal device 10 is created, a stock portion 82 in a right handed version 82a may be imported and connected to a right end of a customized portion 80 and a stock portion 82 in a left handed version 82b may be imported and connected to a left end of the customized portion 80.

Stock portions 82 may be available in different sizes. For example, stock portions 82 may be available in large, medium, and small sizes. The different sizes may correspond to different spacings or gaps between standards 12 and regulators 14. A large size may correspond to a wider gap between a standard 12 and a regulator 14, a smaller size may correspond to a narrower gap between a standard 12 and a regulator 14, etc. Accordingly, when a virtual model of a nasal device 10 is created, a right handed version 82a of a particular size may be imported and connected to a right end of a customized portion 80 and a left handed version 82b of the particular size may be imported and connected to a left end of the customized portion 80.

In selected embodiments, one or both stock portions 82 (e.g., a right handed version 82a, a left handed version 82b) may include a marking 84 (e.g., an “L” for large, an “M” for medium, and an “S” for small) designating a size thereof. Accordingly, when a nasal device 10 is produced (e.g., printed in an additive manufacturing process) the size thereof may be readily apparent.

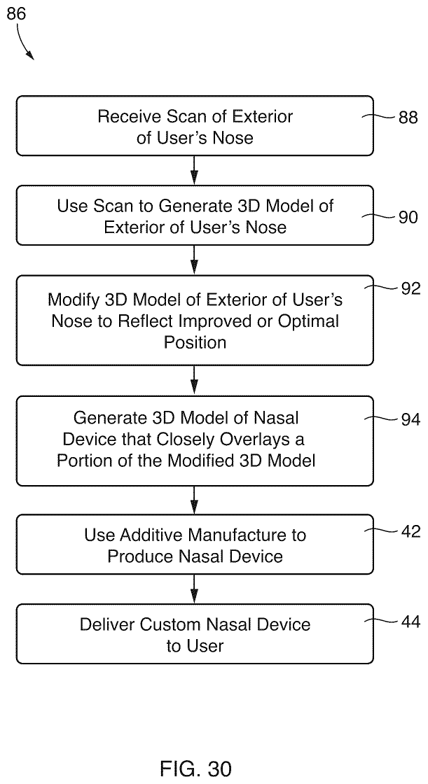

Referring to FIG. 30, in selected embodiments, a method 86 of manufacturing a nasal device 10 may begin with receipt 88 of a digital scan of an exterior of a user's nose. In certain embodiments, a user may use a computerized mobile device (e.g., a smart phone, tablet, or the like) having a camera and running corresponding software to effect the scan. Such a scan may include the user moving a mobile device during the scan. For example, it may include sweeping a mobile device through a horizontal arc of about 180 degrees from one side of his or her face to the other. Thereafter, the user may send the digital scan to a manufacturing entity.

A digital scan may be used to generate 90 a virtual three-dimensional model of a user's nose. Thereafter, this virtual three-dimensional model of a user's nose may be modified 92 or deformed 92 to reflect or portray an improved or optimum breathing position. For example, a technician may use computer software to move (e.g., lift) selected surfaces of the virtual three-dimensional model outward. The selected surfaces may correspond to the flexible tissues 20 on one or both sides of the nose. Accordingly, the original virtual three-dimensional model of a user's nose may correspond to the natural, neutral, or relaxed position of that nose, while the modified virtual three-dimensional model of the user's nose may correspond to an improved or optimum position of the flexible tissues 20 for improved or optimized nasal breathing.

The modified virtual three-dimensional model of the nose may be used to create 94 a virtual three-dimensional model of a nasal device 10. Specifically, a customized portion 80 may be shaped to match the improved or optimized position of the nose 16 of a user. Accordingly, a customized portion 80 may track (e.g., closely follows selected contours of) the nose 16 so positioned. Moreover, one or more stock portions 82 may be digitally defined so as to appropriately fit and extend into corresponding nasal openings 18. For example, a computer-aided drafting (CAD) application or the like may be used to import one or more stock portions 82 into the three dimensional model. The stock portions 82 may be a predefined, standard part. Accordingly, they may be imported and located as desired.

Once a customized portion 80 and one or more stock portions 82 are properly positioned within a virtual three dimensional model, the interfaces between the various components may be filled and/or smoothed so as to provide the desired comfort and functionality. The completed virtual three dimensional model of the nasal device 10 may then be printed 42 in a polymeric material using a three-dimensional printing process. The resulting nasal device 10 may then be delivered 44 to the corresponding user.

In certain embodiments, creating 94 a virtual three-dimensional model of a nasal device 10 may include creating 94 multiple models. For example, in a first model, a customized portion 80 may be connected to two stock portions 82 (e.g., a right handed version 82a and a left handed version 82b) that have a large gap. In a second model, a customized portion 80 may be connected to two stock portions 82 (e.g., a right handed version 82a and a left handed version 82b) that have a medium gap. Finally, in a third model, a customized portion 80 may be connected to two stock portions 82 (e.g., a right handed version 82a and a left handed version 82b) that have a small gap. In such embodiments, three nasal devices 10 (e.g., one with a large gap between a standard 12 and a regulator 14, one with a medium gap between a standard 12 and a regulator 14, and one with a small gap between a standard 12 and a regulator 14) may be additively manufactured 42 and delivered 44 to a user. The user may then try on the various nasal devices 10 and use the one that is most beneficial, comfortable, or the like. The others devices 10 may be discarded. Should the user need a replacement, he or she may just order the size that suited him or her best.

In selected embodiments, an app (e.g., a software application running on a mobile device of a user) may facilitate a method 86 in accordance with the present invention. For example, a user may download an app provided by a manufacturer. Via the app, the user may establish an account. The app may provide information (e.g., text, videos, etc.) that show a user how to perform a digital scan of the exterior of the user's nose 16. The app may also provide a communication link for sending 66 the scan (e.g., one or more computer files or images), payment information, or the like or a combination or sub-combination thereof to the manufacturer.

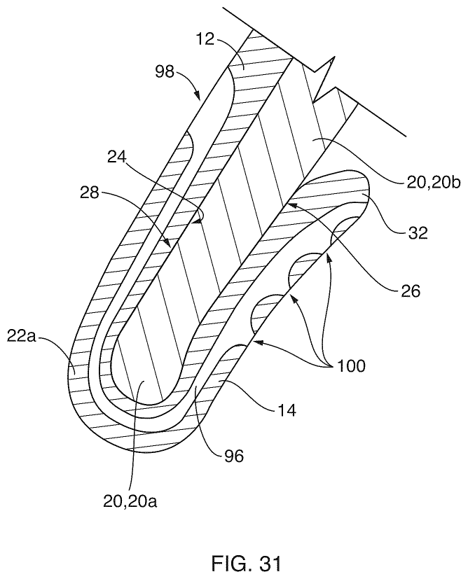

Referring to FIG. 31, in selected embodiments, a nasal device 10 or some portion thereof (e.g., one or more stock portions 82) may include a conduit 96 extending from an exterior aperture 98 (e.g., an aperture 98 located on exterior to the nose 16 of the user) to one or more interior apertures 100 (e.g., one or more apertures 100 located on interior to or proximate an interior of the nose 16 of the user). Liquids (e.g., medications, aroma therapy products, “essential” oils, or the like) applied to an exterior aperture 98 may travel (e.g., wick or move through capillary action) through the conduit 96 and exit (e.g., vaporize) through one or more exit apertures 100. In selected embodiments, wicking or sponge material may be included at the exterior aperture 98, the one or more interior apertures 100, and/or within the conduit 96 to facilitate the absorption, movement, and/or evaporation of the liquids. Accordingly, a nasal device 10 may be a mechanism for delivering medication, fragrances, or the like to a user.

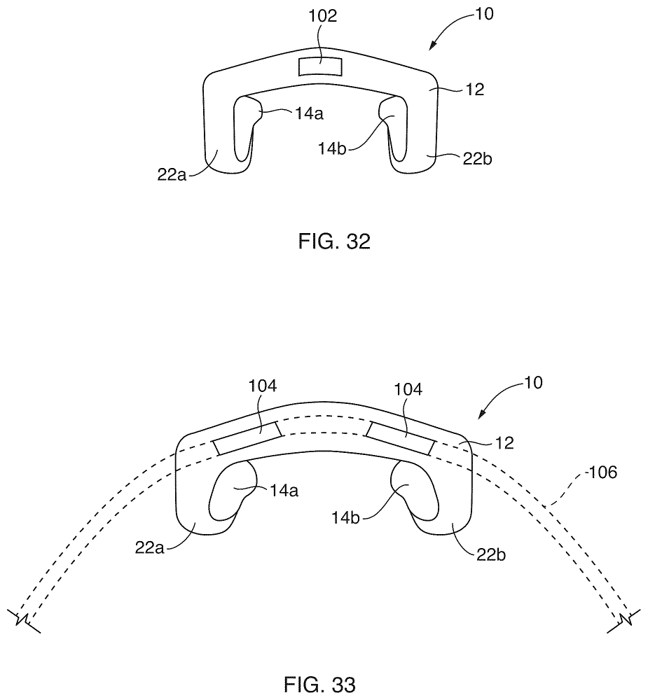

Referring to FIG. 32, in selected embodiments, a nasal device 10 may include a radio frequency temperature sensor 102. In response to being activated by electromagnetic waves, such a sensor 102 may report (e.g., wirelessly transmit) a temperate of the user.

Referring to FIG. 33, in certain embodiments, a nasal device 10 may include one or more engagement mechanisms 104 for receiving and/or securing a rim 106 of a face mask therewithin. In selected embodiments, such a rim 106 may be additively manufactured from a virtual three-dimensional model built to closely and comfortable follow the face of a user. That is, the rim 106 may be built based on a digital scan of the face of the user. Accordingly, a nasal device 10 may assist in securing a mask to a user (e.g., aid one or more elastic or ties in securing the mask to the user) and prevent the mask from deforming the nose of the user in a manner that restricts nasal breathing.

The present invention may be embodied in other specific forms without departing from its spirit or essential characteristics. The described embodiments are to be considered in all respects only as illustrative, and not restrictive. The scope of the invention is, therefore, indicated by the appended claims, rather than by the foregoing description. All changes which come within the meaning and range of equivalency of the claims are to be embraced within their scope.

What is claimed is:

1. A nasal device comprising:
a standard sized and shaped to extend over a portion of an exterior of a nose of a user from a first nasal opening, over flexible tissues of the nose, to a second nasal opening, the standard having a first end proximate the first nasal opening and a second end proximate the second nasal opening;
a first regulator connected to and extending from the first end of the standard and configured to extend into the first nasal opening when the nasal device is applied to the nose of the user;
the first regulator shaped to engage the flexible tissues and regulate a position thereof by pushing the flexible tissues toward an underside of the standard;
a second regulator connected to and extending from the second end of the standard and configured to extend into the second nasal opening when the nasal device is applied to the nose of the user; and
the second regulator shaped to engage the flexible tissues and regulate the position thereof by pushing the flexible tissues toward the underside of the standard.

2. The nasal device of claim 1, wherein the standard, the first regulator, and the second regulator are monolithically formed as a single piece of polymeric material.

3. The nasal device of claim 1, wherein the standard is formed of a polymeric material having a thickness and rigidity sufficient to flex the flexible tissues of the nose.

4. The nasal device of claim 1, wherein the standard is a custom component that matches contours of the nose of the user.

5. The nasal device of claim 1, wherein the standard is a custom component that matches contours of the nose of the user with the flexible tissues of the nose positioned for improved nasal breathing.

6. A nasal device comprising:
a standard sized and shaped to extend over a portion of an exterior of a nose of a user from a first nasal opening to a second nasal opening, the standard having a first end proximate the first nasal opening and a second end proximate the second nasal opening, the standard being a custom component based on a digital scan of the exterior of the nose of the user;
a first regulator connected to and extending from the first end of the standard and configured to extend into the first nasal opening when the nasal device is applied to the nose of the user; and
a second regulator connected to and extending from the second end of the standard and configured to extend into the second nasal opening when the nasal device is applied to the nose of the user.

7. The nasal device of claim 6, wherein the digital scan of the exterior of the nose of the user corresponds to flexible tissues of the nose in a neutral, relaxed position.

8. The nasal device of claim 6, wherein the digital scan of the exterior of the nose of the user corresponds to flexible tissues of the nose positioned for improved nasal breathing.

9. The nasal device of claim 6, wherein the first regulator is shaped to engage flexible tissues of the nose and push the flexible tissues toward an underside of the standard.

10. The nasal device of claim 9, wherein the second regulator is shaped to engage the flexible tissues and push the flexible tissues toward the underside of the standard.

11. The nasal device of claim 6, wherein the standard, the first regulator, and the second regulator are monolithically formed as a single piece of polymeric material.

12. The nasal device of claim 6, wherein the standard is formed of a polymeric material having a thickness and rigidity sufficient to flex flexible tissues of the nose.
